(19) United States
(12) Patent Application Publication
(10) Pub. No.: US 2007/0004021 A1
(43) Pub. Date: Jan. 4, 2007

(54) Plating media for the identification of Yersinia pestis

(76) Inventor: Lawrence Restaino, Elburn, IL (US)

Correspondence Address: MARSHALL A. BURMEISTER, P. O. BOX 765, WILLIAMS BAY, WI, 53191, US

(21) Appl. No.: 11/168,830

(22) Filed: Jun. 28, 2005

Related U.S. Application Data
(60) Provisional application No. 60/583,345, filed on Jun. 28, 2004.

(51) Int. Cl. C12N 1/20

(52) U.S. Cl. 435/253.6

(57) ABSTRACT

A plating media for identifying Yersinia pestis bacteria having nutrients for promoting growth of Yersinia pestis thereby producing beta-glucosidase, carbohydrate that is incapable of reacting with Yersinia pestis, a pH indicator dye, a chromogenic substrate that reacts to beta-glucosidase to form precipitate, and an agent to solidify the mixture, whereby a microorganism which ferments the carbohydrate but does not produce beta-glucosidase will produce colonies of the color determined by the pH indicator dye, Yersinia pestis and other microorganisms that do not ferment the carbohydrate but produce beta-glucosidase activate the substrate to color their colonies with the color of precipitate released by the substrate, and other bacteria which ferment the carbohydrate and produce beta-glucosidase produce colonies of the color that results from mixing the colors described above.

The present invention relates to processes for detecting the presence of Yersinia pestis bacteria in foods, water, humans and animals. It also relates to plating media for the identification and enumeration of bacteria, particularly Yersinia pestis bacteria.

### BACKGROUND OF THE INVENTION

Yersinia pestis is the causative bacteria for the plague or “black death” in man. Plague is one of the oldest known diseases of mankind dating back to A.D. 1348, and it is often fatal. During the 14^{th} century, an epidemic of the plague in Europe caused 25 million deaths. Plague is known to be carried by rodents and spread among humans by fleas that have become infected through contact with infected rodents.

Since 1900, the Unites States has been substantially free of the plague until recent outbreaks among infants. These outbreaks are believed to be caused by Yersinia pestis bacteria present in powdered infant formula. Yersinia pestis is substantially dormant at temperatures below 37 degrees Celsius and can survive for long periods in stored powdered infant formula. Further, once a human being or animal is inoculated with the Yersinia pestis bacteria, plague develops rapidly. Accordingly, there is a pressing need for means to rapidly identify Yersinia pestis bacteria in foods, water, humans and animals. A plating media described in the paper Ber, et al., Development of an Improved Selective Agar Medium for Isolation of Yersinia Pestis, Applied and Environmental Microbiology, 60: 5787-5795 (2003), documents an effort to satisfy this need, but the plating medium of this article requires long incubation times and produces colonies of Yersinia pestis on the medium of the color of the medium, thus making the medium difficult to count and tending to produce many false positives.

### SUMMARY OF THE INVENTION

It is an object of the present invention to provide a plating medium in which Yersinia pestis bacteria will grow and form colonies on the surface of the medium that may be readily observed and enumerated. However, test samples containing Yersinia pestis generally include a plurality of other microorganisms, and it is desirable for plating media to suppress unwanted bacteria and to differentiate colonies of Yersinia pestis from those of other microorganisms. It is known to use various characteristics of bacterial colonies to differentiate the colonies produced on a plating medium by different microorganisms from a mixed sample, including the size, shape, presence of a dome, and the color of the colony. The media of the present invention differentiate Yersinia pestis colonies from the colonies of other bacteria on the basis of contrasting colors.

Culture media containing agar, a carbohydrate and a pH indicator dye have long been used to observe colonies on the surface of the media. The metabolic activity of the microorganisms in the media produces acid which influences the indicator dye and causes the color of the colonies to change. More recently chromogenic substrates have been incorporated in agar medium for coloring colonies of bacteria that utilize the substrate.

It is an object of the present invention to provide a plating medium for identifying and enumerating colonies of Yersinia pestis bacteria that are differentiated by color from the colonies of other microorganisms that are likely to be present in a test sample and cannot be suppressed. The inventor has found that Yersinia pestis will not ferment certain carbohydrates, but is positive with respect to certain substrates. Further, almost all other microorganisms that are likely to be present in a test sample for Yersinia pestis and cannot be suppressed are capable of fermenting a carbohydrate, including Yersinia enterocolitica. The inventor has provided a plating medium including only carbohydrates that are negative for Yersinia pestis and positive for other microorganisms that are likely to be in a test sample, and a substrate that is positive for Yersinia pestis. Since all of the bacteria which cannot be suppressed, other than Yersinia pestis, will not ferment the same carbohydrate, it is desirable to provide a plurality of carbohydrates in the plating media according to the present invention. However, a single carbohydrate may be used in the media of the present invention to differentiate against those bacteria which are positive with respect to that carbohydrate.

The one or more carbohydrates and pH indicator dye, and the substrate of the present invention will display four distinctive colors in a fully processed plate. The first color is the background color of the medium. The second color is the color of colonies that metabolize the substrate but are negative with respect to all carbohydrates, this color being the color of the precipitate released by the substrate. Yersinia pestis bacteria produce colonies of this color. The third color is the color of colonies of unsuppressed bacteria that are positive with respect to one or more carbohydrates and negative with respect to the substrate. Thus the third color is the color produced by the indicator dye. The fourth color is the color of colonies of bacteria that are positive for both the substrate and a carbohydrate, and hence the fourth color is a blend of the colors of the precipitate from the substrate and the indicator dye.

The invention will be more readily understood from the following detailed description of the invention, which has no drawing.

### DETAILED DESCRIPTION OF THE INVENTION

The plating media of the present invention contain nutrients to promote the growth of Yersinia pestis, especially protein. Media containing the following ingredients, either singly or in various mixtures, have been found to be suitable for promoting the growth of Yersinia pestis: tryptone, bacto-peptone, proteose-peptone, meat infusions including heart infusion broth, yeast extract, soytone, and ammonium ferrous sulfate hexahydrate, may be used, but it is to be understood that each of these ingredients can be separately used or used in combination with other nutrients. Further, there are many other nutrients which will support the growth of microorganisms that may be used in place of or in addition to the above named nutrients.

Test samples can be expected to contain bacteria other than Yersinia pestis that may be inhibited or suppressed. Gram positive bacteria and fungi (yeasts) may be inhibited. Such undesired microorganisms may be inhibited by mixing one or more of the following ingredients into the medium: bile salts #3, sodium deoxycholate, irgasan, vancomycin, and cycloheximide. Also, other inhibitors of gram positive bacteria and molds can be employed.

Sodium chloride may be added to the medium for osmolarity purposes.

The detection system for Yersinia pestis of the present invention utilizes a solid plating medium. Preferably agar is added to the other ingredients of the medium to provide the desired mixture for a solid medium. The detection system also uses a pH indicator dye to indicate changes in the pH of the medium.

Media for detecting Yersinia pestis according to the present invention contain at least one and preferably a plurality of carbohydrates, none of which are fermented by the Yersinia pestis bacteria. Suitable carbohydrates are lactose, rhamnose, sucrose, cellobiose, adonitol, inositol, dulcitol, fucose, melezitose, and raffinose.

Closely related bacteria that produce acid from a carbohydrate include the coliforms, such as Escherichia, Klebisella and Enterobacter. Most Citrobacter species and Yersinia enterocolitica produce acid from sucrose, and Salmonella will ferment dulcitol. Most species in the genis Serritia produce acid from adonitol, whereas, Providencia ferments inositol. All four of the above mentioned carbohydrates are not utilized by Yersinia pestis. Further as presently known, the above-identified bacteria do not significantly produce acid from the carbohydrates referred to with respect to other bacteria.

A medium according to the present invention also contains one or more substrates that react to the enzyme beta-glucosidase. Growth of microorganisms in a medium according to the present invention, specifically including Yersinia pestis, can result in production of beta-glucosidase, and the media of the present invention include one or more substrates that react with the beta-glucosidase enzyme. Suitable substrates are 5-bromo-4-chloro-3-indoxyl-beta-D-glucopyranoside, and 3-indoxyl-beta-D-glucopyranoside, which produce an insoluble precipitate that contrasts with the color of the medium and is the second color (with a dark or indigo blue color). In media according to the hereinafter described examples, the presence of colonies of microorganisms that produce beta-glucosidase in the medium and do not produce acids from the carbohydrates, such as Yersinia pestis, are dark or indigo blue and sharply contrast with the color of the plating medium.

While Yersinia pestis is lactose, dulcitol, adonitol, inositol, sucrose, rhamnose and cellobiose negative, other bacteria that are likely to be present in a sample are positive for one or more of these carbohydrates. Microbial fermenting of lactose, dulcitol, adonitol, inositol sucrose rhamnose or cellobiose changes the pH of the medium, thus producing colonies influenced by the indicator dye, a third color. In the following examples this third color is yellow. Hence, the presence of a yellow colony on the surface of an incubated plating medium of the examples is an indication of the presence of a microorganism other than Yersinia pestis. Colonies produced by a microorganism which is both a lactose, dulcitol, adonitol, inositol, sucrose, rhamnose or cellobiose fermenter and beta-glucosidase positive are of a fourth color that is a blend of the second and third colors, namely, green in the following examples.

3-indoxyl-beta-D-glucopyranoside and 5-bromo-4-chloro-3-indoxyl-beta-D-glucopyranoside are chromogens that react to the presence of beta-glucosidase. Beta-glucosidase is an enzyme produced by Yersinia pestis and other enterics, and this enzyme reacts with 3-indoxyl-beta-D-glucopyranoside, or 5-bromo-4-chloro-3-indoxyl-beta-D-glucopyranoside to produce an insoluble indigo blue precipitate. Other chromogens may be used in place of, or in combination with, 3-indoxyl-beta-D-glucopyranoside or 5-bromo-4-chloro-3-indoxyl-beta-D-glucopyranoside. Two such other substrates are 6-chloro-3-indoxyl-beta-D-glucopyronoside and 5-bromo-6-chloro-3-indoxyl-beta-D-glucopyranoside which respond to beta-glucosidase by producing a salmon and majenta precipitate, respectively, in the plating medium that may be observed and counted.

It is desirable to include an ingredient in the medium to enhance the production the production of the beta-glucosidase enzyme. In the following examples of media according to the present invention, isopropyl-beta-D-thioglucopyranoside or 3-O-methyl-beta-D-glucopyranoside is added to the plating medium for this purpose.

### EXAMPLE I

The ingredients for an exemplary embodiment of the plating media of the present invention and the amounts thereof are set forth in the following Table I.

TABLE I

| MATERIAL | MEASUREMENT | |
|---|---|---|
| Tryptone | 5.0 | grams/liter |
| Bacto-peptone | 10.0 | grams/liter |
| Beef (meat) extract | 5.0 | grams/liter |
| Dulcitol | 8.0 | grams/liter |
| Lactose | 8.0 | grams/liter |
| Sucrose | 8.0 | grams/liter |
| Sodium chloride | 5.0 | grams/liter |
| Inositol | 8.0 | grams/liter |
| Cycloheximide | 0.100 | grams/liter |
| 5-bromo-4-chloro-3-indoxyl-beta-D- | 0.200 | grams/liter |
| glucopyranoside | | |
| Phenol red | 0.1 | grams/liter |
| Bile salts #3 | 1.0 | grams/liter |
| 3-indoxyl-beta-D-glucopyranoside | 0.200 | grams/liter |
| Isopropyl-beta-D-thioglucopyranoside | 0.100 | grams/liter |
| Agar | 15.0 | grams/liter |
| Vancomycin | 8.0 | milligrams/liter |

Except for vancomycin, the ingredients are mixed in any order, the pH adjusted to 6.7 to 6.9, boiled, and the mixture is permitted to cool to 50 degrees Celsius. Thereafter, sterile vancomycin is added aseptically to the other ingredients. The composition is then poured into plates, thereafter permitted to dry for 48 to 72 hours in the dark, and then it is ready to be used. Storage time of poured plates is as much as 90 days at 2 to 8 degrees Celsius.

### EXAMPLE II

The ingredients and the amounts thereof of a second example of the present invention are set forth in the following Table II.

TABLE II

| | Supplier | Grams/liter |
|---|---|---|
| Chemical | | |
| Heart Infusion | Difco | 25.00 |
| Broth | | |
| Yeast Extract | Difco | 6.00 |
| Soytone | Difco | 3.13 |
| Ammonium ferrous | Sigma | 0.50 |
| sulfate hexahydrate | (F3754) | |
| Sucrose | Anyplace | 8.00 |
| Rhamnose | Anyplace | 8.00 |
| Cellobiose | Anyplace | 8.00 |
| Lactose | Anyplace | 8.00 |
| 5-Bromo-4-Chloro- | Biosynth AG | 0.20 |
| 3-Indoxyl-β-D- | or Inalco | |
| Gluopyranoside | | |
| 3-O-Methyl-D- | Biosynth AG | 0.15 |
| glucopyranose | | |
| Brom cresol purple | Anyplace | 0.02 |
| Agar | Difco | 15.00 |
| Supplements | | |
| provided by the | | |
| user | | |
| Irgasan | | 0.001 |
| DP 300 (Dissolve | | |
| in 95% ethanol) | | |
| Vancomycin | Sigma | 0.010 |
| hydrochloride | V 2002 | |

Except for irgasan and vancomycin hydrochloride, the ingredients are mixed in any order, the pH adjusted to 7.2 to 7.40, boiled and thereafter the mixture is permitted to cool to 50 degrees Celsius. Thereafter, sterile irgasan and vancomycin hydrochloride are added aseptically to the other ingredients, and the final pH is adjusted to 7.1 to 7.3. The composition is then poured into plates and permitted to dry for 48 to 72 hours in the dark, and then it is ready to be used. Storage time of poured plates is as much as 60 days at 2 to 8 degrees Celsius.

The process of the present invention requires a plate or mass of the plating medium to be inoculated with the test sample, and the inoculated mass is then incubated at a temperature of 28 to 30 degrees Celsius for a period of time to permit growth of the microorganisms in the test sample to observable colonies. If powdered baby formula is to be examined for the presence of Yersinia pestis, it is preferable to mix a small quantity of the powdered baby formula in a nutrient broth, to allow the mixture to incubate for a period of time and to thereafter inoculate the mass of plating media with the liquid mixture. The inventor has found that with the plating media of the examples described above, a period of 48 hours of incubation is sufficient time for Yersinia pestis colonies to grow into colonies that are readily observable with the naked eye. It is believed that the abundant growth of microorganisms in the plating medium of Example I is due to the nutrients provided by the tryptone, bacto-peptone, and meat extract, and the abundant growth of microorganisms in the plating medium of Example II is due to the nutrients provided by the heart infusion broth, yeast extract, soytone and ammonium ferrous sulfate hexahydrate. Finally, the surface of the plating media is assayed and the presence and number of blue with black, or indigo blue, colonies recorded, and these colonies are identified as Yersinia pestis. Also, the presence of white or yellow to green colored colonies is noted as an indication of microorganisms other than Yersinia pestis.

Table III tabulates the growth of a number of microorganisms that are likely to be in a sample to be examined for Yersinia pestis after incubation for a period of 48 hours at 28 to 30 degrees Celsius in the plating media of Example II.

TABLE III

| | Growth on | |
|---|---|---|
| | Yersinia pestis | |
| | Chromogenic | |
| Bacterial strain | Plating Medium | Colony Color |
| Yersinia pestis | + | B |
| Aeromonas hydrophila | + | Y/G |
| Aeromonas veronii | + | G |
| Aeromonoas sobria | + | G |
| Citrobacter freundii | + | Y |
| Edwardsiella tarda | + | Y |
| Enterobacter aerogenes | — | |
| Enterobacter cloacae | — | |
| Generic Escherichia coli | — | |
| Hafnia alvei (2 strains) | — | |
| Klebsiella pneumoniae | + | W |
| Lactobacillus | + | W |
| Listeria monocytogenes | — | |
| Listeria seeligeri | — | |
| Morganella morganii | + | W |
| Proteus mirabilis | — | |
| Providencia rettgeri | + | B/G |
| Pseudomonas aeruginosa | + | W |
| Salmonella spp. (4 strains) | — | |
| Shigella boydii | — | |
| Shigella flexneri | — | |
| Shigella sonnei | — | |
| Vibrio cholerae | — | |
| Vibrio parahaemolyticus | — | |
| Vibrio vulnificus | — | |
| Yersinia enterocolitica (8 | + | Y |
| strains) | | |
| Yersinia enterocolitica 1A | + | G/Y |
| Yersinia aldovae | + | B/G |
| Yersinia fredricksenii | + | G |
| Yersinia intermedia | + | G |
| Yersinia kristensenii | + | Y/G |
| Yersinia pseudotuberculosis (6 | + | G/Y |
| strains) | | |
| Yersinia pseudotuberculosis (7 | + | G |
| strains) | | |

Key:
W = White;
Y = Yellow;
G = Green; and
B = Blue.
+ = Growth and
— = no growth.

It is to be noted that no special equipment is required to observe the incubated surface of the plating medium. The time required observing the number and presence of blue with black colonies is far less than required when other similarly colored colonies are present. Subsequent confirmation of blue with black colonies uses known tests. Also, there are no ingredients in the plating medium that are especially costly. Hence, an assay of a test sample may be made at modest cost.

Those skilled in the art will devise other methods of utilizing the plating media of the present invention and other plating media than those specifically described in the foregoing specification within the scope of the present invention. It is therefore intended that the scope of the present invention be not limited by the foregoing specification, but rather only by the appended claims.

The invention claimed is:

1. A culture plating medium for the presumptive identification of Yersinia pestis from a sample that is also likely to contain other bacteria comprising at least one nutrient for promoting the growth of Yersinia pestis and thus producing the beta-glucosidase enzyme, at least one carbohydrate selected from a group that is capable of being fermented by bacteria other than Yersinia pestis, all carbohydrates in the medium being incapable of being fermented by Yersinia pestis, a pH indicator dye that changes the color of the plating medium from a first color to a second color when the pH of the medium changes, a chromogenic substrate that reacts to the beta-glucosidase enzyme to form precipitate in the plating medium of a third color, and a sufficient mass of an agent to solidify the mixture, whereby a microorganism that achieves growth in the medium but does not metabolize the carbohydrate and does not produce the beta-glucosidase enzyme produces colonies in the medium of the first color, a microorganism which ferments the carbohydrate but does not produce the enzyme beta-glucosidase will produce colonies in the plating medium of the second color, Yersinia pestis and other microorganisms that do not ferment the carbohydrate but produce the enzyme beta-glucosidase activate the substrate to release a precipitate in the medium a the third color, and other bacteria which ferment a carbohydrate and produce the enzyme beta-glucosidase activate the substrate and produce colonies in the plating medium of a fourth color which is the color that results from mixing the second and third colors, the first, second, third and fourth colors contrasting with each other.

2. A culture plating medium for the presumptive identification of Yersinia pestis from a sample that is also likely to contain other bacteria comprising claim 1 wherein the at least one nutrient is selected from the group tryptone, bacto-peptone, proteose-peptone, meat extract including heart infusion broth, yeast extract, soytone, and ammonium ferrous sulfate hexahydrate.

3. A culture plating medium for the presumptive identification of Yersinia pestis from a sample that is also likely to contain other bacteria comprising claim 2 wherein the nutrients comprise tryptone, bacto-peptone and meat extract.

4. A culture plating medium for the presumptive identification of Yersinia pestis from a sample that is also likely to contain other bacteria comprising claim 2 wherein the at least one nutrient comprises heart infusion broth, yeast extract, soytone, and ammonium ferrous sulfate hexahydrate.

5. A culture plating medium for the presumptive identification of Yersinia pestis from a sample that is also likely to contain other bacteria comprising claim 1 wherein the medium contains a plurality of carbohydrates selected from the group lactose, rhamnose, dulcitol, adonitol, inositol, sucrose, cellobiose, fucose, melezitose and raffinose.

6. A culture plating medium for the presumptive identification of Yersinia pestis from a sample that is also likely to contain other bacteria comprising claim 5 wherein the medium contains lactose, dulcitol, inositol, and sucrose.

7. A culture plating medium for the presumptive identification of Yersinia pestis from a sample that is also likely to contain other bacteria comprising claim 5 wherein the medium contains lactose, rhamnose, sucrose, and cellobiose.

8. A culture plating medium for the presumptive identification of Yersinia pestis from a sample that is also likely to contain other bacteria comprising claim 1 wherein the chromogenic substrate is selected from the group 3-indoxyl-beta-D-glucopyranoside, 5-bromo-4-chloro-3-indoxyl-beta-D-glucopyranoside, 6-chloro-3-indoxyl-beta-D-glucopyronoside, and 5-bromo-6-chloro-3-indoxyl-beta-D-glucopyranoside.

9. A culture plating medium for the presumptive identification of Yersinia pestis from a sample that is also likely to contain other bacteria comprising claim 8 wherein the chromogenic substrate comprises 3-indoxyl-beta-D-glucopyranoside, and 5-bromo-4-chloro-3-indoxyl-beta-D-glucopyranoside.

10. A culture plating medium for the presumptive identification of Yersinia pestis from a sample that is also likely to contain other bacteria comprising claim 8 wherein the chromogenic substrate comprises 5-bromo-4-chloro-3-indoxyl-beta-D-glucopyranoside.

11. A culture plating medium for the presumptive identification of Yersinia pestis from a sample that is also likely to contain other bacteria comprising claim 8 and an ingredient in the medium to enhance the production of the beta-glucosidase enzyme comprising a member of the group isopropyl-beta-D-thioglucopyranoside and 3-O-methyl-beta-D-glucopyranoside.

12. A culture plating medium for the presumptive identification of Yersinia pestis from a sample that is also likely to contain other bacteria comprising claim 1 and an ingredient to inhibit growth of gram positive bacteria and fungi.

13. A culture plating medium for the presumptive identification of Yersinia pestis from a sample that is also likely to contain other bacteria comprising nutrients for promoting the growth of Yersinia pestis comprising tryptone, bacto-peptone and meat extract, carbohydrates comprising lactose, dulcitol, inositol, and sucrose, a phenol red pH indicator dye that changes the color of the plating medium from a first color to a second color when the pH of the medium changes, a chromogenic substrate comprising 3-indoxyl-beta-D-glucopyranoside, and 5-bromo-4-chloro-3-indoxyl-beta-D-glucopyranoside for forming a precipitate in the plating medium of a third color, and a sufficient mass of an agent to solidify the mixture, whereby a microorganism that achieves growth in the medium but does not metabolize the carbohydrate and does not produce the beta-glucosidase enzyme produces colonies in the medium of the first color, a microorganism which ferments the carbohydrate but does not produce the enzyme beta-glucosidase will produce colonies in the plating medium of the second color, Yersinia pestis and other microorganisms that do not ferment the carbohydrate but produce the enzyme beta-glucosidase activate the substrate to release a precipitate in the medium a the third color, and other bacteria which ferment a carbohydrate and produce the enzyme beta-glucosidase activate the substrate and produce colonies in the plating medium of a fourth color which is the color that results from mixing the second and third colors, the first, second, third and fourth colors contrasting with each other.

14. A culture plating medium for the presumptive identification of Yersinia pestis from a sample that is also likely to contain other bacteria comprising, nutrients for promoting the growth of Yersinia pestis comprising heart infusion broth, yeast extract, soytone, and ammonium ferrous sulfate hexahydrate, carbohydrates comprising lactose, rhamnose, sucrose, and cellobiose, all carbohydrates in the medium being incapable of being fermented by Yersinia pestis, a brom cresol purple pH indicator dye that changes the color of the plating medium from a first color to a second color when the pH of the medium changes, a chromogenic substrate comprising 5-bromo-4-chloro-3-indoxyl-beta-D-glucopyranoside that reacts with the beta-glucosidase enzyme to produce precipitate of a third color, and a sufficient mass of agar to solidify the mixture, whereby a microorganism that achieves growth in the medium but does not metabolize the carbohydrate and does not produce the beta-glucosidase enzyme produces colonies in the medium of the first color, a microorganism which ferments the carbohydrate but does not produce the enzyme beta-glucosidase will produce colonies in the plating medium of the second color, Yersinia pestis and other microorganisms that do not ferment the carbohydrate but produce the enzyme beta-glucosidase activate the substrate to release a precipitate in the medium a the third color, and other bacteria which ferment a carbohydrate and produce the enzyme beta-glucosidase activate the substrate and produce colonies in the plating medium of a fourth color which is the color that results from mixing the second and third colors, the first, second, third and fourth colors contrasting with each other.
